(19) United States
(12) Patent Application Publication
(10) Pub. No.: US 2008/0175934 A1
(43) Pub. Date: Jul. 24, 2008

(54) NOVEL HERBAL COMPOSITION OF EXTRACTS OF QUERCUS INFECTORIA, PROCESS FOR PREPARING THE SAME AND USE THEREOF

(75) Inventors: Shankar Kumar Mitra, Bangalore (IN); Ekta Saxena, Bangalore (IN); Rangesh Paramesh, Bangalore (IN); Uddagiri Venkanna Babu, Bangalore (IN); Sundaram Ramachandran, Bangalore (IN); Marikunte Venkata Ranganna, Bangalore (IN)

Correspondence Address: DARBY & DARBY P.C., P.O. BOX 770, Church Street Station, New York, NY, 10008-0770, US

(73) Assignee: Himalaya Global Holdings Limited, Grand Cayman, KY (US)

(21) Appl. No.: 11/965,239

(22) Filed: Dec. 27, 2007

(30) Foreign Application Priority Data
Dec. 29, 2006 (IN) 2831/DEL/2006

(51) Int. Cl. A61K 36/49

(52) U.S. Cl. 424/771

(57) ABSTRACT

Disclosed is a novel herbal composition comprising extract of Quercus infectoria having rich and effective concentration of as antioxidants and phenolic compounds. Further the present invention provides a process for preparing said composition and use thereof in cosmeceuticals, pharmaceuticals and health drinks for treating oxidative stress and other life style diseases.

### FIELD OF THE INVENTION

This invention, in general relates to a novel herbal composition comprising extracts of Quercus infectoria. More particularly, the present invention provides a novel herbal composition comprising extract of Quercus infectoria having high and effective concentration of antioxidants and phenolic compounds. The invention further provides a process for preparing said composition and use thereof.

### BACKGROUND OF THE INVENTION

Reactive Oxygen species and Reactive Nitrogen species (ROS/RNS) arise in the normal course of oxidative metabolism. These reactive species (ROS/RNS) influence multiple metabolic, signaling and transcriptional processes, which are involved in the normal function of cell.

Oxidative stress is a condition that occurs when there is excess and prolonged imbalance between the generation of reactive oxygen species and reactive nitrogen species and control by antioxidants. Reducing reactive oxygen species and reactive nitrogen species in the body is one of the primary target towards the protection of various diseases such as aging process, cancer, diabetes and neurodegenerative diseases.

Dietary compounds such as vitamins C and E may limit oxidative damage directly but along these other dietary constituents viz. carotenoids and polyphenols may also act via indirect mechanisms such as endogenous antioxidant enzyme activity and thereby reduce the risk of a number of the age related disorders.

Polyphenols occur most commonly in foods of plant origin have attracted much attention as potent antioxidants due to their ability to scavenge free radical and form inert phenoxy radical intermediates. A number of epidemiological studies have suggested that consumption of polyphenol-rich foods reduces the risk of developing chronic diseases.

Although, these polyphenol antioxidants reported for the prevention of various lifestyle diseases but only a few findings show that they are active and majority of the results show that they have no clinical efficacy. The vast literature on antioxidants suggests that the failure of polyphenol antioxidant in clinical cases because it is almost impossible to eliminate many active oxygen species produced in the body by single antioxidant molecule.

Polyphenol antioxidants derived from the plant origin have structurally phenol group as backbone and make it more lipid soluble. The bioavailability of these plant polyphenols is very low in GI tract and therefore most of these antioxidants have not sufficiently displayed efficacy in the body even though they have been reported as potent antioxidants in vitro.

To overcome the above said difficulties in the preparation of antioxidant composition that is effective both in vitro and clinical, it is necessary to select a group of polyphenolic compounds rich composition that is selectively address the inhibition of many enzymatic pathways that produce free radicals and also the overall composition should improve the bioavailability of the antioxidant active fraction.

### PRIOR ART

U.S. Pat. No. 6,534,046 to Golz-Bemer et al. disclosed the preparation of plant derived anti-perspiration cosmetics comprising the extracts of Equisetum arvense, Salvia officinalis, Hamamelis Virginia and Quercus infectoria.

U.S. Pat. No. 6,264,926 to Farooqi et al. disclose a formulation useful as a natural herbal tooth powder comprising the extracts of Quercus infectoria, Zanthoxylum amatum and Zingiber officinalis etc.

Kaur G et al. (J. Ethnopharmacol. 2004, 90(2-3). 285-92) have reported the anti-inflammatory activities of alcoholic extracts of galls of Quercus infectoria.

Sawangiaroen N et al (J Ethnopharmacol 2005 98(1-2) 67-72) have reported the antidiarrhoel effect of methanol extract of galls of Quercus infectoria.

United States Patent application No. 20030138509 to Pushpangadan et al. teaches the preparation of fermented herbal health drink from the plant Andrographis paniculata.

U.S. Pat. No. 4,741,915 to Farr et al. disclosed the use of purified and hydrolyzed gallotannins derived from plant materials for inhibition of oxidation in foodstuffs.

U.S. Pat. No. 7,041,332 to Gaudout et al. teaches the preparation of a phenolic rich fraction obtained from the fruit of family rosaceae for use in cosmetic, dietary and nutraceutical preparation.

U.S. Pat. No. 6,989,161 to Rouf et al discloses a composition comprising phytonutrients viz. lutein, lycopene, epigallocatechin gallate (EGCG), ellagic acid, hesperidin and quercetin.

### SUMMARY OF THE INVENTION

It is a principal object of the present invention to provide a herbal composition comprising extract of Quercus infectoria having high and effective concentration of antioxidant and phenolic compounds.

Another aspect of the present invention is to obtain the extract using all parts of the plant Quercus Infectoria, preferably from the galls.

Yet another aspect of the present invention is to obtain a herbal composition comprising antioxidant rich extract of Quercus Infectoria, wherein the antioxidant is preferably methyl gallate.

Still another aspect of the present invention is to obtain the extract of Quercus Infectoria employing solvents selected from non-polar hydrocarbon, chlorinated solvents, an ester and ketones, an alcohol and water, preferably, selected from hexane, chloroform, ethyl acetate, acetone, methanol or ethyl alcohol.

Yet another aspect of the present invention is to obtain the extract of the said herbal composition employing methanol with hexane and acetone preferably in 1:1 ratio, wherein the said extract is characterized by having antioxidant concentration of more than 80%.

Still another aspect of the present invention is to obtain the extract of the said herbal composition employing methanol with chloroform and methanol preferably in 1:1 ratio, wherein said extract is characterized by having phenolic compounds concentration of more than about 108 w/w

In accordance with further aspect of the present invention, there is provided an antioxidant and phenolic compounds rich herbal composition, which is obtained by a process, comprising the steps of (a) pulverizing the shade dried galls of Quercus Infectoria to coarse powder, (b) subjecting the resultant coarse powder of said dried galls of Quercus Infectoria into an extraction vessel in presence of a solvent either alone or in combination thereof, (c) filtering the resultant plant extracts employing suitable filter, (d) drying the resultant solvent extracts to form a concentrate.

Further aspect of the present invention is the obtained extract is characterized by having antioxidant principle concentration of more than 80% and phenolic principle concentration of more than about 108% w/w.

In yet another aspect, the present invention is endowed with a process for preparing a herbal composition comprising extract of Quercus Infectoria having high and effective concentration of antioxidant and phenolic principles, alone or in combination with other effective herbs and pharmaceutically acceptable excipients, wherein the said process is comprised of; (a) pulverizing the shade dried galls of Quercus Infectoria to coarse powder, (b) subjecting the resultant coarse powder of said dried galls of Quercus Infectoria into an extraction vessel in presence of a solvent either alone or in combination thereof, (c) filtering the resultant plant extracts employing suitable filter, (d) drying the resultant solvent extracts to form a concentrate, (d) characterizing the resultant extract by analyzing presence of concentration of antioxidant and phenolic compounds, (f) mixing the analyzed resultant extract with pharmaceutically acceptable excipients to prepare the said herbal composition.

It is another aspect of the present invention wherein; the extraction is performed employing any suitable hot or cold extraction techniques, preferably, percolation, maceration or soxhlet method.

One another preferred aspect of the present invention is to effectively incorporate the said herbal composition comprising extract of galls of Quercus Infectoria in cosmetics, pharmaceuticals and functional foods such as energy health drinks to reduce oxidative stress that is associated with ageing, cardiovascular disease, cancer, immunological disorders, dementia, diabetes and macular degeneration and other lifestyle diseases or as natural preservative, antioxidant or as antimicrobial agent.

One another aspect of the present invention is to assess the antioxidant and antimicrobial activity of methanol extract of galls of Quercus Infectoria.

It is yet another aspect of the present invention is to use the said herbal composition for preventing oxidative stress.

It is yet another preferred aspect of the present invention is to evaluate the said herbal composition for antifungal activity employing fungal strains such as Trycophyton rubrum (MTCC296), Candida albicans (MTCC 741), Tricophyton gypseum (ATCC 8125™), Tricophyton mentagrophytes (ATCC 52018).

It is still another preferred aspect of the present invention is to appraise the said herbal composition for antibacterial activity employing bacterial strains such as Escherichia coli (MTCC 443), Pseudomonas aeruginosa (MTCC 741), Salmonella typhi (MTCC 733), Staphylococcus aureus (MTCC 96), Helicobacter pylori (ATCC No-51653™), Niesseria gonorrhoeae (ATCC_{—}49226).

Another aspect of the present invention is to evaluate the inhibitory activity of said herbal composition against yeast Malassezia furfur (MTCC-1374) causing dandruff and Malassezia pachydermatis causing ear infection.

In one another preferred aspect of the present invention is to evaluate the antioxidant and free radical scavenging activity of the said herbal composition by employing DPPH method, Ferric reducing Power Assay Method, and determining Nitric oxide scavenging activity.

Yet another aspect of the present invention is to prepare the extracts of Quercus Infectoria by employing percolation method and hot soxhlet method.

Another aspect of the present invention is to prepare the various extracts such as methanol extract, water extract, acetone extract, ethyl acetate and acetone (1:1) extract, chloroform and methanol (8:2) extract in order to obtain 20% of the raw materials.

It is another aspect of the present invention is to identify the various active fractions of the methanolic extract of Quercus infectoria with varying degree of activity.

In one another aspect of the present invention is to estimate the percentage concentration of methyl gallate present in the various active fractions of Quercus infectoria by HPLC standardization.

It is still another aspect of the present invention is to estimate the presence of total phenolic compounds in various solvent fractions and semi purified fractions of extract of Quercus infectoria.

One another aspect of the present invention is to employ the column chromatography for purifying the active compounds of various active fractions of Quercus infectoria.

Additional aspect of the present invention is to prepare an anti stress health drink comprising water extract of Withania somnifera (1.0 g), Emblica officinalis (1.0 g), Vitis vinifera (3.0 g) and methanol extract of galls of Quercus infectoria (AXT-3) (0.6 g).

Further aspect of the present invention is to prepare the energy health drink comprising the water extracts of Camellia sinensis (6.0 g.), Withania somnifera (2.0 g), Emblica officinalis (2.0 g), Vitis vinifera (6.0 g) and methanol extract of galls of Quercus infectoria (AXT-3) (0.6 g.)

### BRIEF DESCRIPTION OF DRAWINGS

Further objects of the present invention together with additional features contributing thereto and advantages accruing there from will be apparent from the description of preferred embodiments of the present invention which are shown in the accompanying drawing figures.

FIG. 1. DPPH radical scavenging activity of Quercus infectoria extracts (IC₅₀-[μg/ml] values) AXT-01, 02, 16, 17, 18.

FIG. 2. DPPH radical scavenging activity of Quercus infectoria extracts (IC₅₀-[μg/ml] values) AXT-03, 04, 05, 06, 07, 08, 09, 10, 11, 12, 13, 14, 15, 21, 24.

FIG. 3. DPPH radical scavenging activity of Quercus infectoria extracts (IC₅₀-[μg/ml] values) AXT-22, 23.

FIG. 4. DPPH radical scavenging activity of Quercus infectoria extracts (IC₅₀-[μg/ml] values) AXT-19, 20.

FIG. 5. Reducing power assay of Quercus infectoria extracts (IC₅₀-[μg/ml] values) AXT-01, 02, 16, 17, 18.

FIG. 6. Reducing power assay of Quercus infectoria extracts (IC₅₀-[μg/ml] values) AXT-03, 04, 05, 06, 07, 08, 09, 10, 11, 12, 13, 14, 15, 21, 24.

FIG. 7. Reducing power assay of Quercus infectoria extracts (IC₅₀-[μg/ml] values) AXT-22, 23.

FIG. 8. Reducing power assay of Quercus infectoria extracts (IC₅₀-[μg/ml] values) AXT-19, 20.

FIG. 9. Lipid peroxidation (in vitro) assay of Quercus infectoria extracts (IC₅₀-[μg/ml] values) AXT-01, 03, 10, 31, 33.

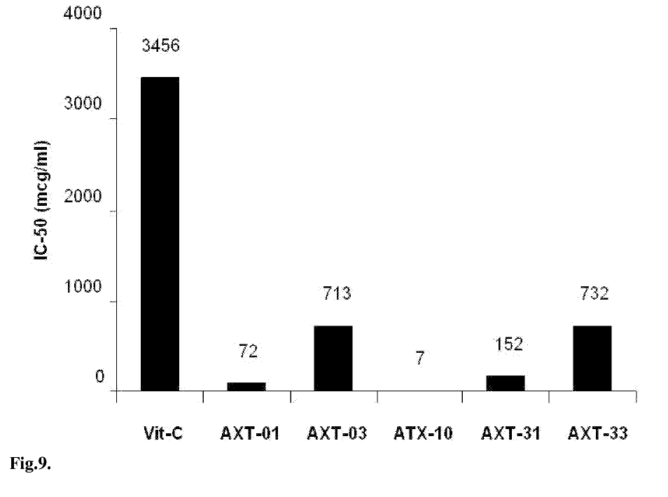

FIG. 10. NO (Nitric Oxide) scavenging activity of Quercus infectoria extracts (IC₅₀-[μg/ml] values) AXT-03, 05, 07, 09, 11, 16, 17, 19, 20, 21, 23, 28, 30, 31.

### DETAILED DESCRIPTION OF THE INVENTION

The present invention involves the selection of herb and subjecting the herb for solvent extraction by various methods and screening of these solvent extracts for the antioxidant activity and antimicrobial activity. Preparation of a composition using the bioactive extract for pharmaceuticals, cosmeceuticals and health drinks as an antioxidant to treat and prevent various life style disease such as stress, ageing, cardiovascular disease, cancer, immunological disorders, dementia, diabetes and macular degeneration and also prevents microbial contamination as natural preservative.

Quercus infectoria of the family Fagaceae is a small tree or shrub and about 2-5 m. high, leaves are very rigid, 4-6 cm.long, very rigid, often glabrescent with spinous teeth, mostly distributed in the temperate regions of the northern hemisphere and extending to sub-tropical and tropical America and Asia at high altitudes. Around, 23 evergreen are found in the Himalayan region of India

Gall nut is used externally for its astringent effect; it is used in ointments for the treatment of piles, and in plasters. The tannic and gallic acids extracted from the galls are often used in dysentery and diarrhoea and as a gargle.

### EXAMPLE-1

### Preparation of Quercus infectoria Extract by Percolation Method

The shade dried galls of Quercus infectoria was pulverized to coarse powder and about 1 Kg each of powdered material placed in different percolators and the material was soaked in n-hexane, dichloromethane, chloroform, ethyl acetate, acetone, ethanol, methanol and water either alone or in combination thereof at room temperature for 24 to 48 h then plant extracts were drained out from the percolator and filtered through whatmann no. 1 filter paper. The percolation of the residual material was again carried out with respective solvents and the combined solvent extracts concentrated to dryness on rotatory evaporator or on steam bath at optimum temperature and under reduced pressure.

### EXAMPLE-2

### Preparation of Quercus infectoria Extract by Hot-Soxhlation Method

The shade dried galls of Quercus infectoria was pulverized to coarse powder and about 1 Kg each of the powdered material subjected to hot-soxlation in different soxhlet apparatus using solvents n-hexane, dichloromethane, chloroform, ethyl acetate, acetone, ethanol and methanol either alone or combination thereof at optimum temperature until extraction is completed, then plant extracts were filtered through whatmann no. 1 filter paper and concentrated to dryness on rotatory evaporator or on steam bath at optimum temperature. All extracts were qualitatively similar to extracts prepared by percolation method. The yields of solvent extracts that are considerably more than 20% are summarized in the table-1.

TABLE 1

| Sl | | | | |
|---|---|---|---|---|
| No | Code No | Solvents Used | Source | Yield (%) |
| 1 | AXT-1 | Methanol | Plant material | 82.0 |
| 2 | AXT-2 | Water | Plant material | 60.0 |
| 3 | AXT-16 | Ethyl acetate:Acetone (1:1) | Plant material | 63.3 |
| 4 | AXT-17 | Chloroform:Methanol (8:2) | Plant material | 21.5 |
| 5 | AXT-18 | Acetone | Plant material | 69.5 |

### EXAMPLE-3

### Solvent-Solvent Fractionation of Methanol Extract (AXT-1)

About 1 Kg of methanol extract was macerated with different solvents alone or combination thereof as mentioned in the table-2 to obtain a purified active fraction with potent antioxidant and antimicrobial activities. The yields of active fractions obtained from the solvent-solvent fractionation are given in table-2.

TABLE 2

| Sl | Code | | | Yield |
|---|---|---|---|---|
| No | No | Solvents Used | Source | (%) |
| 1 | AXT-3 | Hexane:acetone(1:1) | Methanol extract | 7.0 |
| 2 | AXT-4 | Hexane:acetone (6:4) | Methanol extract | 4.0 |
| 3 | AXT-5 | Chloroform:methanol (9:1) | Methanol extract | 7.5 |
| 4 | AXT-6 | Chloroform:methanol (1:1) | Methanol extract | 70.0 |
| 5 | AXT-7 | Dichloromethane:acetone (7:3) | Methanol extract | 2.5 |
| 6 | AXT-8 | Dichloromethane:acetone (1:1) | Methanol extract | 46.3 |
| 7 | AXT-9 | Chloroform:acetone (1:1) | Methanol extract | 4.2 |
| 8 | AXT-10 | Ethyl acetate:acetone (1:1) | Methanol extract | 88.2 |
| 9 | AXT-11 | Toluene:methanol (9:1) | Methanol extract | 2.0 |
| 10 | AXT-12 | Ethyl acetate | Methanol extract | 69.2 |
| 11 | AXT-13 | Ethyl acetate:methanol (7.5:2.5) | Methanol extract | 94.0 |
| 12 | AXT-14 | Ethyl acetate:methanol (1:1) | Methanol extract | 96.0 |
| 13 | AXT-15 | Ethyl acetate:acetone (6:4) | Methanol extract | 80.0 |
| 14 | AXT-21 | Dichloromethane:methanol (1:1) | Methanol extract | 82.0 |
| 15 | AXT-24 | Ethyl acetate:acetone (2.5:7.5) | Methanol extract | 88.0 |
| 16 | AXT-25 | Acetone | Methanol extract | 80 |

### EXAMPLE-4

### Solvent-Solvent Fractionation of Water Extract (AXT-2)

About 1 Kg each of water extract was suspended in dichloromethane:acetone (7:3) and dichloromethane:acetone (1:1) to obtain semipurified fractions AXT-22 and AXT-23 respectively. The yields of AXT-22 and AXT-23 fractions are given in table-3.

TABLE 3

| Sl | | | | Yield |
|---|---|---|---|---|
| No | Code No | Solvents Used | Source | (%) |
| 1 | AXT-22 | Dichloromethane:acetone (7:3) | Water extract | 5.0 |
| 2 | AXT-23 | Dichloromethane:acetone (1:1) | Water extract | 8.0 |

### EXAMPLE-5

### Solvent-Solvent Fractionation of Semipurified Ethyl Acetate Fraction (AXT-12)

The ethyl acetate fraction obtained at the yield of 69.2% from the methanol extract was further fractionated with dichloromethane:acetone (7:3) and dichloromethane:acetone (1:1) to yield AXT-19 and AXT-20 respectively. The yields of these fractions are given in table-4

TABLE 4

| | Code | | | |
|---|---|---|---|---|
| Sl No | No | Solvents Used | Source | Yield (%) |
| 1 | AXT-19 | Dichloromethane:acetone(7:3) | Ethyl acetate | 9.0 |
| | | | fraction | |
| 2 | AXT-20 | Dichloromethane:acetone(1:1) | Ethyl acetate | 10.0 |
| | | | fraction | |

### EXAMPLE-6

### Column Chromatography of Methanol Extract (AXT-1)

The column chromatography was performed over silica gel (60-120 mesh). About 2.5 Kg of silica gel was suspended in chloroform and packed the column. About 300 g of methanol extract was prepared as slurry using silica gel and poured on to the column. The column was then eluted with chloroform, chloroform:methanol with increasing polarity to obtain purified active fraction. The details of column fractions and their yields are summarized in the table-5.

TABLE 5

| Sl. No. | Fraction No | Solvent system | Yield (g) |
|---|---|---|---|
| 1 | AXT-1/1 | Chloroform | 5 |
| 2 | AXT-1/2 | Chloroform:methanol (95:5) | 7 |
| 3 | AXT-1/3 | Chloroform:methanol (90:10) | 50 |
| 4 | AXT-1/4 | Chloroform:methanol (85:15) | 15 |
| 5 | AXT-1/5 | Chloroform:methanol (80:20) | 100 |
| 6 | AXT-1/6 | Methanol | 100 |

### EXAMPLE-7

### Standardization of Extracts and Fractions by TLC and HPLC

All extracts and fractions were prepared at a concentration of 100 mg in 5 mL of the respective solvent and about 50 μl of the sample solution were spotted on precoated (e-Merck) silica gel TLC plates. The TLC plates were air dried and placed in the mobile Phase: (1) Dichloromethane:Methanol (85:15) and (2) Hexane:Ethyl acetate (30:70). TLC plates were air dried and sprayed with 1% alcoholic ferric chloride solution to visualize as dark blue spots confirming the presence of phenolic compounds.

All extracts and fractions were also subjected to HPLC analysis for standardization purpose taking methyl gallate as principle marker. The results are summarized in table-6. The HPLC conditions are as follows.

| | |
|---|---|
| Column: | Reverse Phase C-18 |
| Mobile Phase: | Solvent A:Solvent B (20:80) |
| Solvent A: | 0.05% phosphoric acid in ACN |
| Solvent B: | 0.1% phosphoric acid in water |
| Flow rate: | 1 ml/min |
| Detection: | UV λₘₐₓ 240 nm |

TABLE 6

| Sl No | Code No | (% of Methyl gallate) |
|---|---|---|
| 1 | AXT-1 | 11.6 |
| 2 | AXT-2 | 0.2 |
| 3 | AXT-3 | 79.9 |
| 4 | AXT-4 | 70.0 |
| 5 | AXT-5 | 64.7 |
| 6 | AXT-6 | 30.4 |
| 7 | AXT-7 | 58.9 |
| 8 | AXT-8 | 37.7 |
| 9 | AXT-9 | 48.0 |
| 10 | AXT-10 | 32.4 |
| 11 | AXT-11 | 72.4 |
| 12 | AXT-12 | 27.3 |
| 13 | AXT-13 | 21.5 |
| 14 | AXT-14 | 28.8 |
| 15 | AXT-15 | 30.0 |
| 16 | AXT-16 | 7.5 |
| 17 | AXT-17 | 15.9 |
| 18 | AXT-18 | 6.0 |
| 19 | AXT-19 | 58.3 |
| 20 | AXT-20 | 36.5 |
| 21 | AXT-21 | 31.8 |
| 22 | AXT-22 | 64.6 |
| 23 | AXT-23 | 77.8 |
| 24 | AXT-24 | 30.2 |
| 25 | AXT-25 | 25.8 |

### EXAMPLE-8

### Quantitative Estimation of Phenolic Compounds

The phenolics present in the extracts and fractions of Quercus infectoria was quantitatively measured using tanninc acid as standard. The greenish blue colour produced during reaction of phenolic compounds with potassium ferri cyanide and ferric chloride was measured at 720 nm. The results are summarized in the table-7.

TABLE 7

| Sl. | | Total phenols |
|---|---|---|
| No | Code No | (Tannic acid) w/w |
| 1 | AXT-1 | 90.09 |
| 2 | AXT-2 | 74.05 |
| 3 | AXT-3 | 97.72 |
| 4 | AXT-4 | 90.45 |
| 5 | AXT-5 | 82.13 |
| 6 | AXT-6 | 108.31 |
| 7 | AXT-7 | 87.22 |
| 8 | AXT-8 | 97.02 |
| 9 | AXT-9 | 91.91 |
| 10 | AXT-10 | 83.96 |
| 11 | AXT-11 | 96.37 |
| 12 | AXT-12 | 98.53 |
| 13 | AXT-13 | 83.97 |
| 14 | AXT-14 | 80.2 |
| 15 | AXT-15 | 89.52 |
| 16 | AXT-16 | 78.22 |
| 17 | AXT-17 | 92.91 |
| 18 | AXT-18 | 92.20 |
| 19 | AXT-19 | 98.55 |
| 20 | AXT-20 | 93.65 |
| 21 | AXT-21 | 94.71 |
| 22 | AXT-22 | 96.33 |
| 23 | AXT-23 | 94.40 |
| 24 | AXT-24 | 96.32 |
| 25 | AXT-25 | 93.04 |

### EXAMPLE-9

### MIC Assay for Antifungal Activity by Agar Dilution Method

The test organisms Trycophyton rubrum (MTCC 296) and Candida albicans (MTCC 741) were procured from IMTECH, Chandigarh, India and Tricophyton gypseum (ATCC 8125™), Tricophyton mentagrophytes (ATCC 52018), were procured from USA. These are dermatophytes causing infections of hair, nails and skin in humans. These are cosmopolitan in distribution

Preparation of Test Samples (Extracts) for Screening

About 100 mg of test sample (extract) was weighed in to a sterile screw capped tube and dissolved in 1 ml of sterile distilled water in case of water soluble extracts, otherwise, extracts were dissolved in 100 μL of DMSO and then subsequently in 900 μL of sterile distilled water.

Media Preparation

Sabouraud chloremphenicol Agar (Himedia) was used for cultivation of fungal strains. The sabouraud chloremphenicol (65 grams) was suspended in 1 Litre distilled water, heated to boil to dissolve the media completely and autoclaved at 15 lbs at 121° C. for 15 minutes.

Inoculum Preparation

A seven day old pure culture of the fungi was taken for the test.

Candida albicans—The 24 hrs culture of Candida albicans was suspended from agar plate into 2 ml of sterile saline. The turbidity of the suspension was adjusted to McFarland's turbidometer standard 0.5.

Procedure

The required amount of the test sample was taken in the petri plate and mixed it with 4 ml of the molten sabouraud chloremphenicol Agar. (To achieve 1 mg/ml concentrations in the plate dilute 40 μL of the extract (100 mg/ml). A range of concentrations with the extracts in duplicate was prepared. A plate for negative control (media control), a plate for positive control without extract were saved and allowed to solidify. The plates were marked on the bottom for the names of the organisms viz. Trycophyton rubrum, Tricophyton gypseum, and Tricophyton mentagrophytes. A small amount of mycelia were picked with sterile inoculation stab and inoculated on the surface of the plate. The plates were incubated at room temp for 7 days and observed for visible growth and MIC were calculated. The results are summarized in the table-8

### EXAMPLE-10

### MIC Assay for Antibacterial Activity

The test organisms Escherichia coli (MTCC 443), Pseudomonas aeruginosa (MTCC 741), Salmonella typhi (MTCC 733), Staphylococcus aureus (MTCC 96) were obtained from IMTECH, Chandigarh, India.

Preparation of Samples for Screening

About 100 mg of test sample (extract) was weighed in to a sterile screw capped tube and dissolved in 1 ml of sterile distilled water in case of water soluble extracts, otherwise, extracts were dissolved in 100 μl of DMSO and then subsequently in 900 μl of sterile distilled water.

Media Preparation

Mueller Hinton Agar (Himedia M173, 38 g.) was suspended in 1 Litre of distilled water and heated to dissolve the medium completely. Mueller Hinton Broth (Himedia M391) 21 g. was suspended in 1 Litre distilled water and autoclaved at 15 lbs pressure at 121° C. for 15 minutes. Miller Hinton Agar was cooled to 55° C.

### Inoculum Preparation

The overnight culture of test bacteria was suspended from the agar plate into 2 ml of sterile saline and adjusted the turbidity of the suspension to McFarlands turbidometer standard 0.5. Dilution ( 1/100^{th}) of the adjusted inoculum was prepared in Muller Hinton Broth. This inoculum contained approximately 1\tilde{×}1^{□} Organisms per ml.

Procedure

The required amount of the extract was taken in the petri plate and mixed it with 4 ml of the molten Mueller Hinton agar. (To achieve 1 mg/ml concentration in the plate diluted 40 μl of extract (100 mg/ml). A range of concentrations was prepared with the extracts in duplicates. A plate for negative control (media control), and a plate for positive control without extract were kept to solidify. Inoculate was spotted with 20 μl of adjusted inoculum on the surface of the agar plate. The plates were incubated for 48 hours at 37° C. Observed the plates for visual growth and MIC were calculated. The results are given in table-8.

### EXAMPLE-11

### Assay for Gram-Negative Rod Helicobacter pylori (ATCC No-51653™)

Media Preparation

Mueller Hinton Agar (Himedia M173) 38 g was suspended in 1 Litre of distilled water. Tryptic soya broth (Himedia) 21 g was put in 1 Litre distilled water. About 10% defibrinated sheep blood was autoclaved at 15 lbs pressure at 121° C. for 15 minutes. These were autoclaved separately. Autoclaved 10% defibrinated sheep blood was added to miller Hinton Agar. The medium was brought to 55° C.

Inoculum Preparation

The fully grown test bacteria (5 days) was suspended from the agar plate into 2 ml of sterile saline and adjust the turbidity of the suspension to McFarlands turbidometer standard 0.5. Prepared 1/100^{th} dilution of the adjusted inoculum in Tryptic soya Broth. This inoculum contain approximately X Organisms per ml.

Procedure

The required amount of the extract was taken in the petriplate and mixed it with 4 ml of the molten Mueller Hinton agar+10% defibrinated sheep blood. (To achieve 1 mg/ml concentration in the plate diluted 40 micro liter of extract (100 mg/ml). A range of concentrations were prepared with the extracts in duplicates. A plate for negative control (media control), and a plate for positive control without extract were kept to solidify. Inoculate was spotted with 20 μl of adjusted inoculum on the surface of the agar plate. The plates were incubated for 5 days in anaerobic jar at 37° C. Observed the plates for visual growth and MIC were calculated. The results are given in table-8.

### EXAMPLE-12

### Assay for Gram-Negative Cocci Niesseria gonorrhoeae (ATCC 49226)

Media Preparation

GC Agar Base (Himedia) (7.2 g) was prepared in distilled water (100 ml) to make a double strength base. Heated to boiling to dissolve the medium completely. The same was sterilized by autoclaving at 15 lbs pressure (121° C.) for 15 minutes. And allowed to cool up to 55° C. aseptically. Separately prepared Haemoglobin (FD002 Himedia) (2%) was added. The vitamin Growth supplements (FD025 Himedia) were added to increase the selectivity of the medium.

Inoculum Preparation

About 48 hours incubated culture kept at 37° C. with 5% CO₂ was suspended in to 2 ml of sterile saline. The inoculum was adjusted to McFarland standard 0.5. Prepared 1/100^{th} dilution of this in Trypric Soya Broth (DIFCO).

Procedure

The media was kept at 55° C. on hot plate to keep molten. The required amount of test sample (extract) was taken in sterile 50 mm petri plate and labeled with the name and concentration of the drug. The test sample was diluted with molten medium in duplicates (To get final concentration of the drug at 1 mg/ml dissolve 40 μL of the extract in 4 ml of the molten medium) One plate for positive control without drug and a plate for media control were kept. The plates were allowed to solidify and then were inoculated with 20 μL of the adjusted inoculum incubated for 48 hours at 37° C. in a anaerobic chamber with 5% CO₂. [According to instructions of Gas pack (BBL)]. After incubation plates were observed for visual growth by comparing with control. Results were recorded. MIC were calculated as the lowest concentration of the drug showing no growth. The results are summarized in table-8.

### EXAMPLE-13

### MIC Assay for Yeast

The test organisms Malassezia furfur (MTCC-1374) is lipophilic yeast found on skin and body surface. The organism was used to test antidandruff activity. This is procured from IMTECH, Chandigarh, India.

Media Preparation

Sabouraud Dextrose Agar-Emmons modified (Himedia) (23.5 g) was suspended in 500 ml distilled water. Heated to boil to dissolve the medium completely and sterilized by autoclaving at 15 lbs pressure, 121° C. for 15 minutes. After cooling by bringing to 55° C. added few drops of sterile corn oil and mixed the medium

Inoculum Preparation

The fresh culture of M. furfur (incubated for 4 or 5 days) from the Agar plate was suspended into 2 ml of sterile saline. The turbidity of the suspension was adjusted to McFarlands turbidometer standard 0.5.

Procedure

Sterile petriplates (50 mm) were labeled with the test sample number and percentage of the sample. The required amount of the test sample was added into petriplates and the molten medium to make up the volume to 4 ml in duplicates. A plate as negative control without inoculation of test organism, and a plate as positive control with test organism were saved. The contents were mixed gently and allowed to solidify. Adjusted inoculate 20 microlitre was spotted on the surface of the media plates. The plates were incubated for 7 days at room temp and observed for visible growth by comparing with positive control. MIC were calculated and results given in table-8.

### EXAMPLE-14

### MIC Assay for Yeast Malassezia pachydermatis

The test organism Malassezia pachydermatis is yeast causing ear infections in canines (MTCC-1369) procured from IMTECH Chandigarh, India. The organism was used to test ear infections.

Media Preparation

Sabouraud Dextrose Agar-Emmons modified (Himedia) (23.5 g) was suspended in 500 ml distilled water. Heated to boil to dissolve the medium completely and sterilized by autoclaving at 15 lbs pressure, 121° C. for 15 minutes. After cooling by bringing to 55° C. added few drops of sterile corn oil and mixed the medium uniformly.

Inoculum Preparation

The fresh culture of M. pachydermatis (incubated for 7 days) from the agar plate was suspended into 2 ml of sterile saline. The turbidity of the suspension was adjusted to McFarlands turbidometer standard 0.5.

Procedure

Sterile petriplates (50 mm) were labeled with the test sample number and percentage of the sample. The required amount of the test sample was added into petriplates and the molten medium to make up the volume to 4 ml in duplicates. A plate as negative control without inoculation of test organism, and a plate as positive control with test organism were saved. The contents were mixed gently and allowed to solidify. Adjusted inoculate 20 μL was spotted on the surface of the media plates. The plates were incubated for 7 days at room temp and observed for visible growth by comparing with positive control. MIC (mg/ml conc) were calculated and results are given in table-8.

TABLE 8

| Organisms | | | | | | | | | | | | |
|---|---|---|---|---|---|---|---|---|---|---|---|---|
| Tested | AXT-5 | AXT-7 | AXT-8 | AXT-9 | AXT-10 | AXT-11 | AXT-17 | AXT-19 | AXT-20 | AXT-21 | AXT-22 | AXT-23 |
| Neisseria | 0.025 | 0.050 | 0.100 | 0.050 | 0.050 | 0.050 | 0.100 | 0.050 | 0.100 | 0.100 | 0.025 | 0.050 |
| gonorrhoeae | | | | | | | | | | | | |
| Trichophyton | 2 | 2 | 2 | 2 | 2 | 1 | 2 | 2 | 2 | 1 | 2 | 2 |
| rubrum | | | | | | | | | | | | |
| Trichophyton | 2 | 2 | 1 | 1 | 2 | 2 | 2 | 1 | 1 | 1 | 2 | 2 |
| mentagrophytes | | | | | | | | | | | | |
| Microsporum | 2 | 2 | 1 | 1 | 2 | 2 | 2 | 2 | 1 | 1 | 2 | 2 |
| gypseum | | | | | | | | | | | | |
| Entameba | 0.250 | 0.250 | 0.200 | 0.200 | 0.250 | 0.250 | 0.200 | 0.200 | 0.250 | 0.250 | 0.200 | 0.200 |
| Coli | | | | | | | | | | | | |
| Staphylococcus | 0.500 | 0.450 | 0.450 | 0.500 | 0.450 | 0.500 | 0.450 | 0.450 | 0.500 | 0.450 | 0.500 | 0.500 |
| aureus | | | | | | | | | | | | |
| Salmonella | 0.250 | 0.200 | 0.500 | 0.500 | 0.250 | 0.200 | 0.500 | 0.500 | 0.500 | 0.150 | 0.150 | 0.150 |
| Typhi | | | | | | | | | | | | |
| Pseudomonas | 2 | 0.500 | 1 | 1 | 2 | 2 | 2 | 1 | 1 | 1 | 2 | 2 |
| aeruginosa | | | | | | | | | | | | |
| Helicobacter | 0.500 | 0.500 | 0.500 | 1 | 1 | 2 | 2 | 2 | 0.500 | 1 | 1 | 1 |
| pylori | | | | | | | | | | | | |
| Candida | 0.250 | 0.200 | 0.500 | 0.500 | 0.250 | 0.200 | 0.500 | 0.500 | 0.500 | 0.150 | 0.150 | 0.150 |
| albicans | | | | | | | | | | | | |
| Mallethsia | 0.500 | 0.450 | 0.450 | 0.500 | 0.450 | 0.500 | 0.450 | 0.450 | 0.500 | 0.450 | 0.500 | 0.500 |
| Furfur | | | | | | | | | | | | |
| Mallathesia | 0.500 | 0.450 | 0.450 | 0.500 | 0.450 | 0.500 | 0.450 | 0.450 | 0.500 | 0.450 | 0.500 | 0.500 |
| Pachydermatis | | | | | | | | | | | | |

### EXAMPLE-15

### Antioxidant Activity of Extracts and Fractions of Quercus Infectoria

All extracts and fractions prepared from the galls of Quercus infectoria was screened for antioxidant activity in three in vitro models viz. DPPH methods, reducing power assay and NO scavenging activity and one ex vivo Lipid peroxidation assay methods.

### EXAMPLE-16

### Free Radical Scavenging Activity by DPPH Method

The free radical scavenging activity of the test sample was measured in terms of hydrogen donating or radical-scavenging ability using the stable radical DPPH.

Procedure

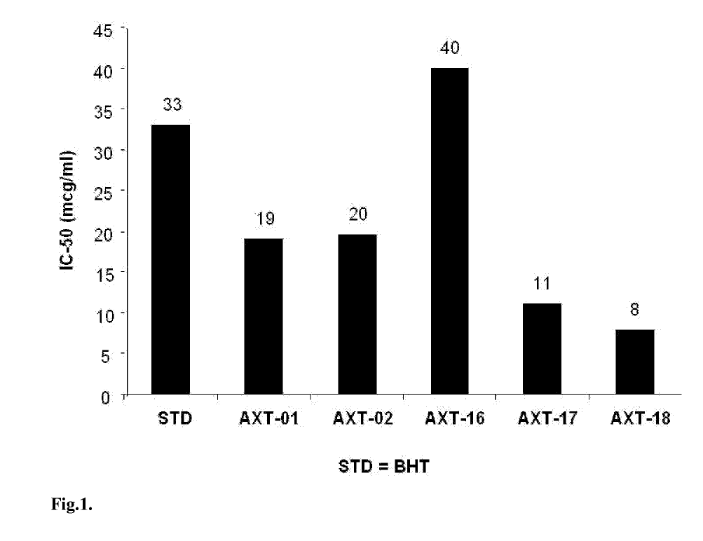

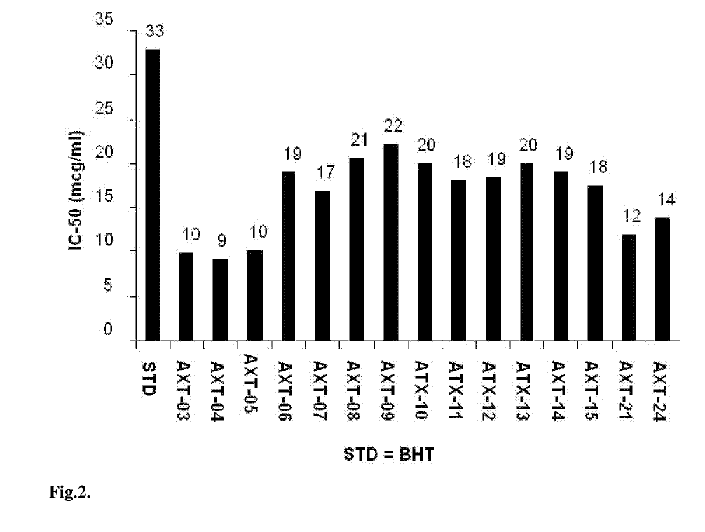

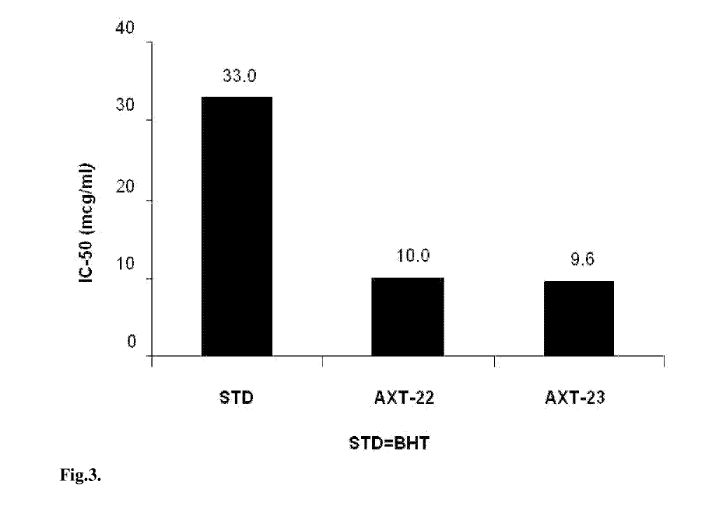

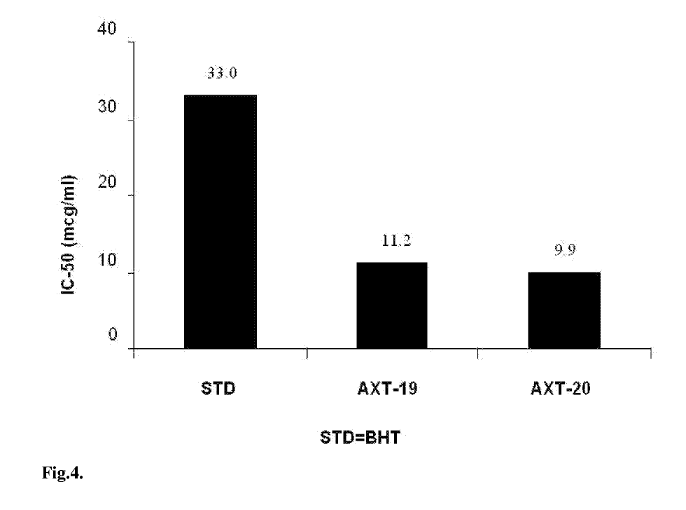

1 ml of 0.1 mM solution of DPPH in ethanol was added to 3.0 ml of test sample solution in water at different concentrations (The stock sample solutions was serially diluted with respective solvents to obtain lower dilutions). Thirty minutes later, the absorbance was measured at 517 nm. Lower absorbance of the reaction mixture was indicated higher free radical scavenging activity. Butylated hydroxy toluene (BHT) was used as standard. The capability to scavenge the DPPH radical was calculated. The antioxidant activity of the test samples was expressed as IC₅₀. The IC₅₀ value is defined as the concentration (in μg/ml) of extracts that inhibited the formation of DPPH radicals by 50%. The results of are shown in FIG. 1-4

### EXAMPLE-17

### Free Radical Scavenging Activity by Reducing Power Assay

The reducing power of test sample (extract) was determined in Fe³⁺—Fe²⁺ redox system according to the method of Oyaizu (1986).

Procedure

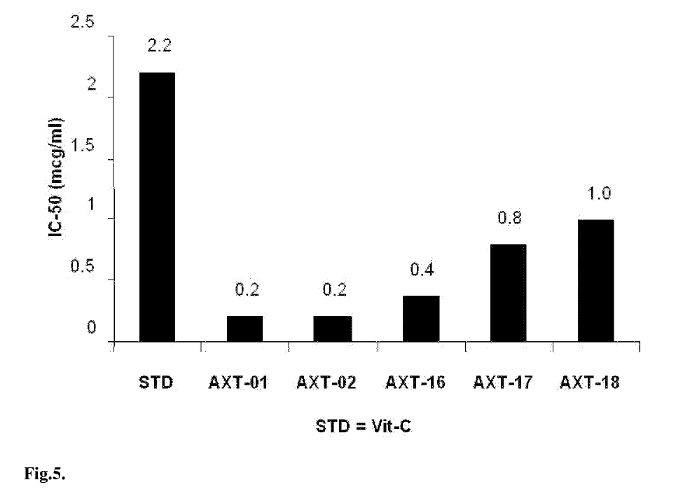

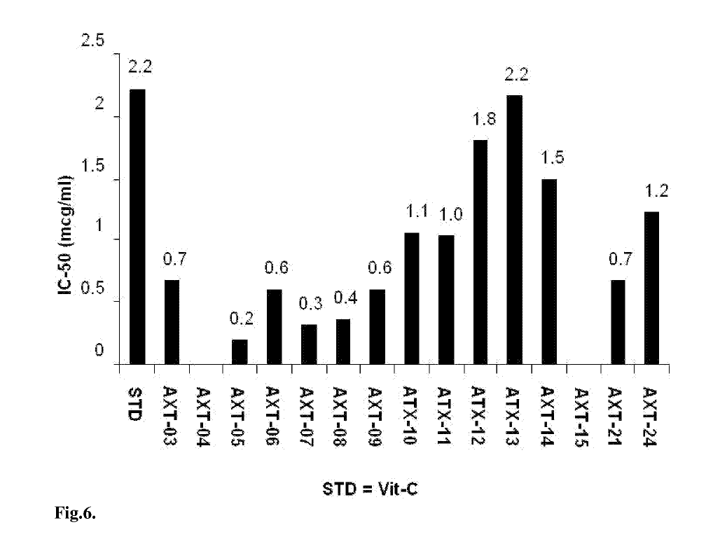

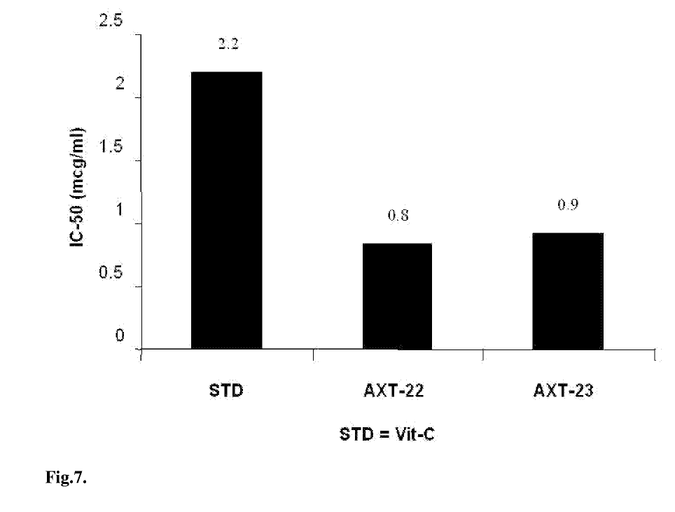

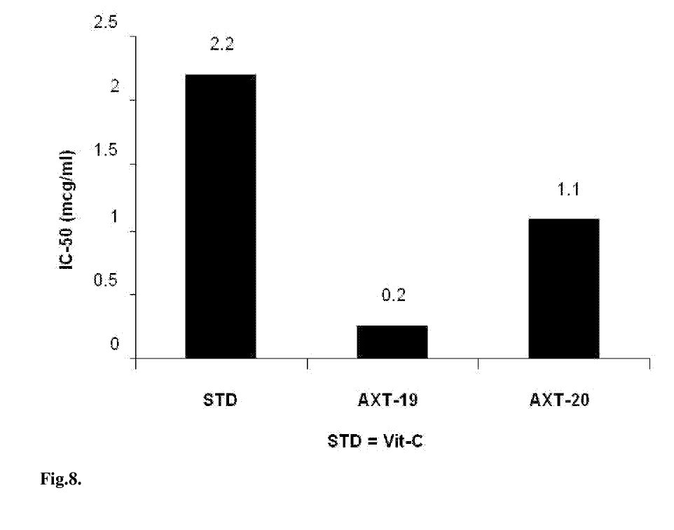

Various concentrations of the test samples (extracts) (The stock test sample solutions was serially diluted with respective solvents to obtain lower dilutions) in 1.0 ml of deionized water was mixed with phosphate buffer (2.5 ml, 0.2M, pH 6.6) and 1% potassium ferricyanide (2.5 ml). The mixture was incubated at 50° C. for 20 min. Aliquots of trichloroacetic acid (2.5 ml, 10%) was added to the mixture, which was then centrifuged at 1036×g for 10 min. The upper layer of solution (2.5 ml) was mixed with distilled water (2.5 ml) and a freshly prepared FeCl₃ solution (0.5 ml, 0.1%). The absorbance was measured at 700 nm. Increased absorbance of the reaction mixture indicated increased reducing power. The results are shown in FIG. 5-8.

### EXAMPLE-18

### Scavenging Activity Against Nitric Oxide

### Nitrite Detection Method

Sodium nitroprusside in aqueous solution at physiological pH spontaneously generates nitric oxide, which interacts with oxygen to produce nitrite ions that can be estimated by use of Greiss reagent. Scavengers of nitric oxide compete with oxygen leading to reduce production of nitric oxide.

Procedure

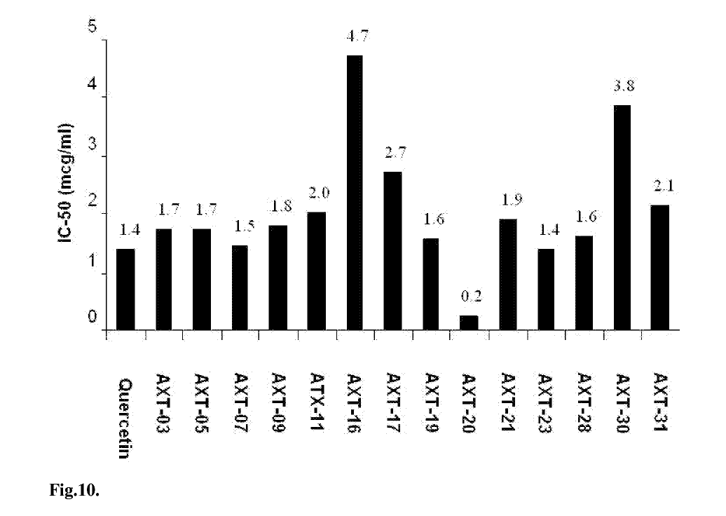

1.0 ml Sodium nitroprusside (5 mM) in 20 mM phosphate-buffered saline (PBS) pH 7.4 was mixed with 1.0 ml of different concentrations of test samples (extracts) (The stock test sample solutions was serially diluted with respective solvents to obtain lower dilutions) and incubated at 25° C. for 150 min. The samples from the above were reacted with 1 ml of Greiss reagent. The absorbance of the chromophore formed during the diazotization of nitrite with sulphanilamide and subsequent coupling with napthylethylenediamine was read at 546 nm and referred to the absorbance of standard solutions of potassium nitrite, treated in the same way with Griess reagent. The percentages of NO scavenging activity of test samples (extracts) are shown in FIG. 10.

### EXAMPLE-19

All herbal antistress drink were prepared using the said antioxidant composition, AXT-3 along with water extracts of Withania somnifera, Emblica officinalis, Vitis vinifera, Sugar syrup, Citric acid, Ascorbic acid and Sodium benzoate as per the formula given below.

| | | | |
|---|---|---|---|
| 1. | Withania somnifera | 1.0 | g |
| 2. | Emblica officinalis | 1.0 | g |
| 3. | Vitis vinifera | 3.0 | g |
| 4. | AXT-3 | 0.6 | g |
| 5. | Citric acid | 15.0 | g |
| 6. | Ascorbic acid | 2.4 | g |
| 7. | Sodium benzoate | 1.0 | g |
| 8. | Sugar syrup | 1.0 | L |
| 9. | Flavour (passion fruit) | 10.0 | g |
| 10. | DM water (q.s.) | 2.0 | L |

### EXAMPLE-20

All herbal Energy drink was prepared using the said antioxidant composition AXT-3 along with water extracts of Camellia sinensis, Withania somnifera, Emblica officinalis, Vitis vinifera, Sucrose, Citric acid, thickening agent (stimuleol) as per the formula given below.

| | | | |
|---|---|---|---|
| 1. | Camelia sinensis | 6.0 | g |
| 2. | Withania somnifera | 2.0 | g |
| 3. | Emblica officinalis | 2.0 | g |
| 4. | Vitis vinifera | 6.0 | g |
| 5. | AXT-3 | 0.6 | g |
| 6. | Citric acid | 3.0 | g |
| 7. | Sucrose | 125.0 | g |
| 8. | Stimuleol | 0.5 | g |
| 9. | Mixed fruit flavour | 1.0 | g |
| 10. | DM water (q.s.) | 1.0 | L |

While this invention has been described in detail with reference to certain preferred embodiments, it should be appreciated that the present invention is not limited to those precise embodiments. Rather, in view of the present disclosure, which describes the current best mode for practicing the invention, many modifications and variations would present themselves to those skilled in the art without departing from the scope and spirit of this invention.

We claim:

1. A Herbal composition comprising extract of Quercus infectoria having high and effective concentration of antioxidant and phenolic compounds, alone or in combination with other effective herbs and pharmaceutically acceptable excipients.

2. The composition according to claim 1, wherein said extract is obtained from all parts of the plant Quercus infectoria, preferably from galls.

3. The composition according to claim 1, wherein said antioxidant is preferably methyl gallate.

4. The composition according to claim 1, wherein said extract is obtained by using solvent selected from non-polar hydrocarbon, chlorinated solvent, an ester, ketone, an alcohol or water.

5. The composition according to claim 4, wherein the solvent is preferably selected from hexane, chloroform and dichloromethane, ethyl acetate, acetone, methanol and ethyl alcohol.

6. The herbal composition according to claim 5, wherein said extract is obtained by fractionation of methanol extract with hexane and acetone (1:1) solvent mixture and wherein said fraction is characterized by having antioxidant concentration of more than about 80%.

7. The herbal composition according to claim 5, wherein said extract is obtained by fractionation of methanol extract with chloroform and methanol (1:1) solvent mixture.

8. The composition according to claim 1, wherein said composition is effectively incorporated in cosmetics, pharmaceuticals and functional foods such as energy health drinks to reduce oxidative stress that is associated with ageing, cardiovascular disease, cancer, immunological disorders, dementia, diabetes or macular degeneration and other lifestyle diseases or as natural preservative or as antimicrobial agent.

9. The herbal composition according to claim 1, wherein said composition is effectively used as antistress herbal drinks.

10. The herbal composition according to claim 9, wherein said antistress herbal drink composition comprises water extracts of Withania somnifera (1.0 g), Emblica officinalis (1.0 g), vitis vinifera (3.0 g) and methanol extract of Quercus infectoria (0.6 g).

11. The herbal composition according to claim 1, wherein said composition is effectively used as energy health drinks.

12. The herbal composition according to claim 11, wherein said energy health drink composition comprises water extracts of Camellia sinensis (6.0 g), Withania somnifera (2.0 g), Emblica officinalis (2.0 g), vitis vinifera (6.0 g) and methanol extract of Quercus infectoria (0.6 g).

13. The herbal composition according to claim 1, wherein said extract is obtained by a process comprising steps of:
(a) pulverizing the shade dried galls of Quercus infectoria to coarse powder,
(b) subjecting the resultant coarse powder of said dried galls of Quercus infectoria into an extraction vessel in presence of a solvent either alone or in combination thereof,
(c) filtering the resultant plant extracts employing suitable filter,
(d) drying the resultant solvent extracts to form a concentrate,
wherein obtained extract is characterized by having antioxidant concentration of more than about 80% and is used in the preparation of said herbal composition.

14. A process for preparing a herbal composition comprising extract of Quercus infectoria having high and effective concentration of antioxidant and phenolic compounds, alone or in combination with other effective herbs and pharmaceutically acceptable excipients, the process comprising;
(a) extracting said extract by pulverizing the shade dried galls of Quercus infectoria to coarse powder,
(b) subjecting the resultant coarse powder of said dried galls of Quercus infectoria into an extraction vessel in presence of a solvent either alone or in combination thereof,
(c) filtering the resultant plant extracts employing suitable filter,
(d) drying the resultant solvent extracts to form a concentrate, and a thick oil portion from said concentrate,
(e) characterizing the resultant extract by analyzing the concentration of antioxidants and phenolic compounds in the resultant and,
(f) mixing the analyzed resultant extract with pharmaceutically acceptable excipients to prepare said herbal composition.

15. The process according to claim 14, wherein extraction is performed employing any extraction technique.

16. The process according to claim 14, wherein extraction is performed preferably employing percolation, maceration or soxhlet method.

17. The process according to claim 14, wherein the solvent used in the process is selected from non-polar hydrocarbon, chlorinated solvent, an ester, ketone, an alcohol or water.

18. The process according to claim 17, wherein the solvent used in the process is preferably selected from hexane, chloroform and dichloromethane, ethyl acetate, acetone, methanol or ethyl alcohol.

19. The process according to claim 18, wherein said solvent is preferably methanol for extraction and followed by hexane and acetone (1:1) solvent mixture for fractionation.

20. The process according to claim 14, wherein obtained extract is characterized by having antioxidant concentration of more than about 80%.

21. The process according to claim 14, wherein said solvent is preferably methanol for extraction and followed by chloroform and methanol (1:1) solvent mixture for fractionation.

22. The process according to claim 14, wherein obtained extract is characterized by having phenolic compounds concentration potential of more than about 108 w/w against tannic acid.

23. A herbal composition comprising extract of Quercus infectoria having high and effective concentration of antioxidant and phenolic compounds, alone or in combination with other effective herbs and pharmaceutically acceptable excipients, wherein said extract is obtained by a process comprising steps of:
(a) pulverizing the shade dried galls of Quercus infectoria to coarse powder,
(b) subjecting the resultant coarse powder of said dried galls of Quercus infectoria into an extraction vessel in presence of a solvent either alone or in combination thereof,
(c) filtering the resultant plant extracts employing suitable filter,
(d) drying the resultant solvent extracts to form a concentrate.

24. The composition according to claim 23, wherein the solvent is selected from non-polar hydrocarbon, chlorinated solvent, an ester, ketone, an alcohol or water.

25. The composition according to claim 24, wherein the solvent is preferably selected from hexane, chloroform and dichloromethane, ethyl acetate, acetone, methanol or ethyl alcohol.

26. The composition according to claim 23, wherein said solvent is preferably methanol with hexane and acetone in 1:1 ratio.

27. The composition according to claim 26, wherein obtained extract is characterized by having antioxidant concentration of more than about 80%.

28. The composition according to claim 23, wherein said solvent is preferably methanol with chloroform and methanol in 1:1 ratio.

29. The composition according to claim 23, wherein obtained extract is characterized by having phenolic compounds concentration of more than about 108 w/w.

30. The composition according to claim 23, wherein said composition is effectively incorporated in cosmetics, pharmaceuticals and functional foods such as energy health drinks to reduce oxidative stress that is associated with ageing, cardiovascular disease, cancer, immunological disorders, dementia, diabetes and macular degeneration and other lifestyle diseases or as natural preservative or as antimicrobial agent.
